(19) United States
(12) Patent Application Publication
(10) Pub. No.: US 2025/0104867 A1
(43) Pub. Date: Mar. 27, 2025

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventor: Yohei MOMOKI, Tokyo (JP)

(21) Appl. No.: 18/893,974

(22) Filed: Sep. 24, 2024

(30) Foreign Application Priority Data
Sep. 27, 2023 (JP) 2023-166220

(51) Int. Cl. G16H 50/20; G16H 30/20; G16H 50/70

(52) U.S. Cl. CPC G16H 50/20; G16H 30/20; G16H 50/70

(57) ABSTRACT

An information processing apparatus including at least one processor, wherein the processor is configured to: search for a plurality of comment-on-findings candidates related to a search query from a comment-on-findings group including a plurality of comments on findings; evaluate each of the comment-on-findings candidates based on importance predetermined for each combination of diagnostic information and finding information included in the comment-on-findings candidates; and present at least one of the comment-on-findings candidates based on a result of the evaluation.

### CROSS-REFERENCE TO RELATED APPLICATIONS

This application claims priority from Japanese Application No. 2023-166220, filed on Sep. 27, 2023, the entire disclosure of which is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. A creator of an interpretation report, such as a radiologist, interprets medical images and creates an interpretation report including a comment on findings.

In order to support the creation of an interpretation report, a method has been proposed that searches for and presents an appropriate comment on finding from a created past comment-on-findings group. For example, JP2008-146220A discloses a method for executing a process of searching for a structured document database based on word-based input information by a user and executing a process of generating a sentence describing a morphological description of an image based on the input information.

In addition, for example, JP2006-155002A discloses a method for searching for and outputting a reference interpretation report from among a plurality of interpretation reports created at the time of past interpretation and expressed in a structured tag format, using input tag information as a search key.

In a case of searching for a comment on findings, it is desirable to use a search query as concise as possible so that a desired comment on findings can be quickly found. On the other hand, the more concise the search query, the greater the number and variations of comments on findings presented as search results, making it more difficult for a user to quickly find a desired comment on findings. Therefore, there is a demand for a technology that can quickly find a desired comment on findings even with a concise search query.

### SUMMARY

The present disclosure provides an information processing apparatus, an information processing method, and an information processing program capable of searching for an appropriate comment on findings with a concise search query.

According to a first aspect of the present disclosure, there is provided an information processing apparatus comprising at least one processor, in which the processor is configured to: search for a plurality of comment-on-findings candidates related to a search query from a comment-on-findings group including a plurality of comments on findings; evaluate each of the comment-on-findings candidates based on importance predetermined for each combination of diagnostic information and finding information included in the comment-on-findings candidates; and present at least one of the comment-on-findings candidates based on a result of the evaluation.

In the first aspect, the processor may be configured to rearrange and present the plurality of comment-on-findings candidates in order of the importance evaluated as being high.

In the first aspect, the processor may be configured to: divide the plurality of comment-on-findings candidates into a plurality of groups based on a type of the diagnostic information included in the comment-on-findings candidates; and present, for each group, at least one of the comment-on-findings candidates.

In the first aspect, the processor may be configured to rearrange and present, for each group, the plurality of comment-on-findings candidates in order of the importance evaluated as being high.

In the first aspect, the processor may be configured to present, for each group, one of the comment-on-findings candidates that is evaluated as having the highest importance.

In the first aspect, the processor may be configured to: further divide the comment-on-findings candidates included in the groups into subgroups based on a predetermined criterion; and present, for each subgroup, at least one of the comment-on-findings candidates.

In the first aspect, the predetermined criterion may be at least one of the number of types of the diagnostic information included in the comment-on-findings candidates or factuality of the diagnostic information included in the comment-on-findings candidates.

In the first aspect, the processor may be configured to rearrange the groups corresponding to the respective pieces of diagnostic information in descending order of a frequency with which the diagnostic information is included in the comment-on-findings group.

In the first aspect, the importance may be a value corresponding to a frequency with which the combination is included in the comment-on-findings group.

In the first aspect, the importance may be a value corresponding to a degree of uniqueness with which the finding information included in the combination is not included in combinations with other diagnostic information.

In the first aspect, the importance may be a value corresponding to a frequency with which the combination is included in the comment-on-findings group and a degree of uniqueness with which the finding information included in the combination is not included in combinations with other diagnostic information.

In the first aspect, the processor may be configured to, in a case in which the comment-on-findings candidates include a plurality of different combinations, perform the evaluation based on the importance for each of the combinations.

In the first aspect, the importance may be predetermined for each combination included in the comment-on-findings group, and the processor may be configured to, in a case in which the comment-on-findings candidate includes the combination for which the importance is not predetermined, correct the evaluation of the comment-on-findings candidate to be lowered.

In the first aspect, the processor may be configured to, in a case in which there are a plurality of the comment-on-findings candidates that include the same combination and have the same result of the evaluation, select and present any one of the comment-on-findings candidates.

In the first aspect, the processor may be configured to, in a case in which there are a plurality of the comment-on-findings candidates that include the same combination and have the same result of the evaluation, select and present one having the smallest sentence volume among the comment-on-findings candidates.

In the first aspect, the processor may be configured to receive an input of the search query by a user.

In the first aspect, the processor may be configured to: acquire an image; and generate at least one of the finding information or the diagnostic information for the image as the search query based on the image.

In the first aspect, the finding information may indicate at least one of a type, a property, a position, a measurement value, and a number of a region of interest, a phrase expressing a change in the region of interest, or a type of diagnostic information other than the diagnostic information to be paired.

According to a second aspect of the present disclosure, there is provided an information processing method executed by a computer, the information processing method comprising: searching for a plurality of comment-on-findings candidates related to a search query from a comment-on-findings group including a plurality of comments on findings; evaluating each of the comment-on-findings candidates based on importance predetermined for each combination of diagnostic information and finding information included in the comment-on-findings candidates; and presenting at least one of the comment-on-findings candidates based on a result of the evaluation.

According to a third aspect of the present disclosure, there is provided an information processing program for causing a computer to execute: searching for a plurality of comment-on-findings candidates related to a search query from a comment-on-findings group including a plurality of comments on findings; evaluating each of the comment-on-findings candidates based on importance predetermined for each combination of diagnostic information and finding information included in the comment-on-findings candidates; and presenting at least one of the comment-on-findings candidates based on a result of the evaluation.

With the information processing apparatus, the information processing method, and the information processing program according to the above aspects of the present disclosure, it is possible to search for an appropriate comment on findings with a concise search query.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is a diagram showing an example of information registered in a report DB.

FIG. 7 is a diagram showing an example of an importance table.

FIG. 8 is a diagram showing an example of a comment-on-findings candidate to be searched for.

FIG. 9 is a diagram showing an example of a screen displayed on a display.

FIG. 10 is a diagram showing an example of a screen displayed on a display.

FIG. 11 is a flowchart showing an example of information processing.

### DETAILED DESCRIPTION

Hereinafter, embodiments of the present disclosure will be described with reference to the drawings.

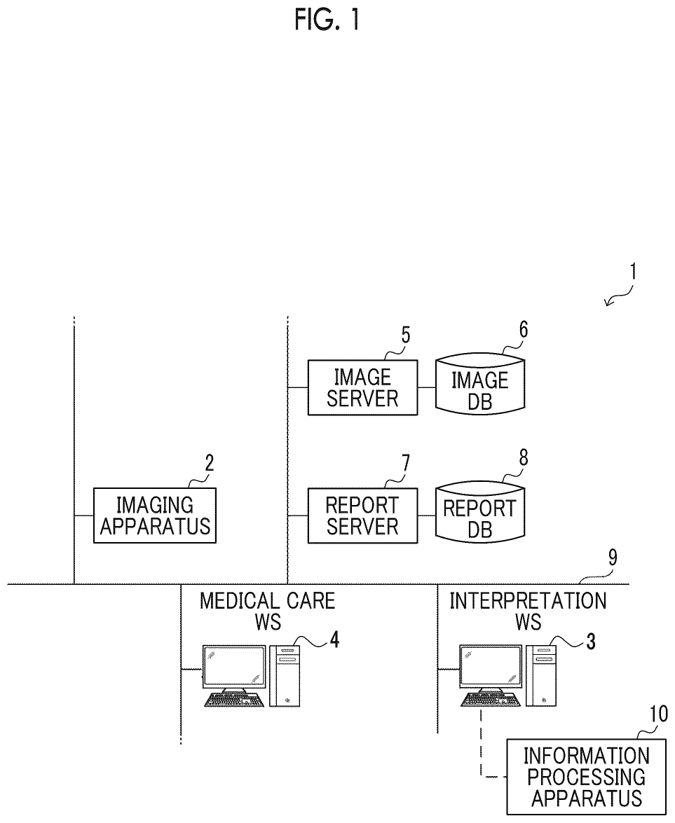

First, a configuration of an information processing system 1 to which an information processing apparatus 10 according to the present disclosure is applied will be described with reference to FIG. 1. FIG. 1 is a diagram showing a schematic configuration of the information processing system 1. The information processing system 1 performs imaging of an examination target part of a subject and storing of the captured medical images based on an examination order from a doctor in a medical department using a known ordering system. In addition, the information processing system 1 allows a radiologist to interpret a medical image and create an interpretation report, and allows a doctor of a medical department that is a request source to view the interpretation report.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc read-only memory (DVD-ROM) or a compact disc read-only memory (CD-ROM), and distributed, and be installed on the computer from the recording medium. In addition, the application program may be stored in, for example, a storage device of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Examples of the imaging apparatus 2 include a simple X-ray imaging apparatus, a computed tomography (CT) apparatus, a magnetic resonance imaging (MRI) apparatus, a positron emission tomography (PET) apparatus, an ultrasound diagnostic apparatus, an endoscope, a fundus camera, and the like. The medical image generated by the imaging apparatus 2 is transmitted to the image server 5 and is stored in the image DB 6.

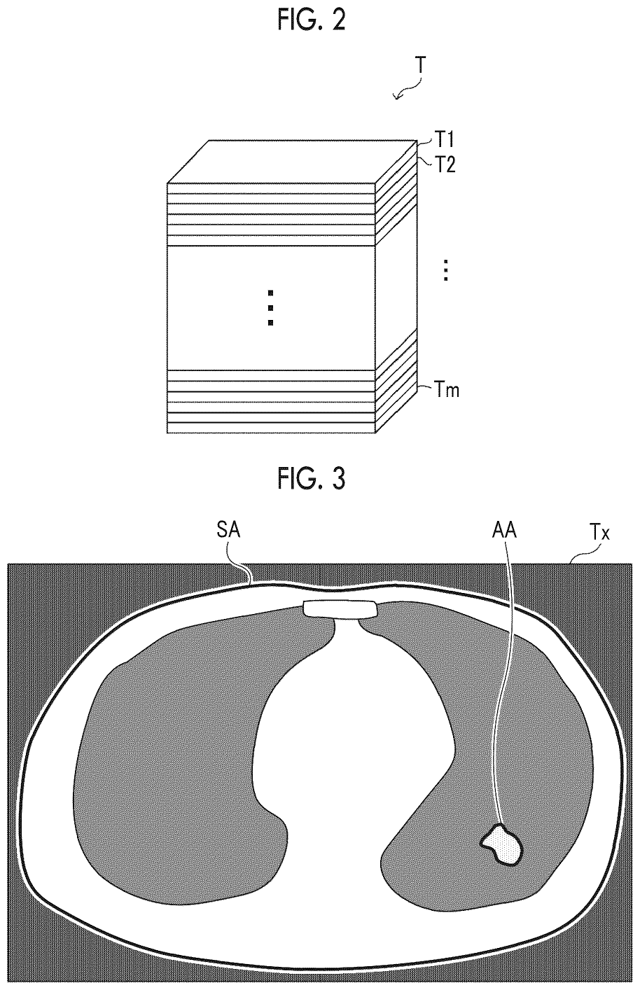

FIG. 2 is a diagram schematically showing an example of a medical image acquired by the imaging apparatus 2. A medical image T shown in FIG. 2 is, for example, a CT image consisting of a plurality of tomographic images T1 to Tm (m is 2 or more) representing tomographic planes from the head to the waist of one subject (human body). The medical image T is an example of an image according to the present disclosure.

FIG. 3 is a diagram schematically showing an example of one tomographic image Tx out of the plurality of tomographic images T1 to Tm. The tomographic image Tx shown in FIG. 3 represents a tomographic plane including lungs. Each of the tomographic images T1 to Tm may include a region SA of a structure showing various organs and viscera of the human body (for example, lungs, kidneys, and the like), various tissues constituting various organs and viscera (for example, blood vessels, nerves, muscles, and the like), and the like. In addition, each tomographic image may include a lesion region AA such as, for example, nodules, tumors, injuries, defects, and inflammation. In the tomographic image Tx shown in FIG. 3, the lung region is the region SA of the structure, and the nodule region is the lesion region AA. A single tomographic image may include regions SA of a plurality of structures and/or lesion regions AA. Hereinafter, at least one of the region SA of the structure included in the medical image or the lesion region AA included in the medical image will be referred to as a “region of interest”.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various types of image processing for the medical image received from the image server 5, display of the medical image, and input reception of a sentence regarding the medical image are performed. In the interpretation WS 3, analysis processing for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing device, a display device such as a display, and input devices such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid-state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, an imaging purpose, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, date of birth, age, and gender of the subject.

In a case in which the image server 5 receives a request to register a medical image from the imaging apparatus 2, the image server 5 prepares the medical image in a format for a database and registers the medical image in the image DB 6. In addition, in a case in which the viewing request from the interpretation WS 3 and the medical care WS 4 is received, the image server 5 searches for a medical image registered in the image DB 6 and transmits the found medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered (details will be described later).

Further, in a case in which the report server 7 receives a request to register the interpretation report from the interpretation WS 3, the report server 7 prepares the interpretation report in a format for a database and registers the interpretation report in the report DB 8. Further, in a case in which the report server 7 receives the viewing request for the interpretation report from the interpretation WS 3 and the medical care WS 4, the report server 7 searches for the interpretation report registered in the report DB 8, and transmits the found interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

Meanwhile, in a case in which the medical image is interpreted and the interpretation report is created in the interpretation WS 3, a past interpretation report (comment on findings) registered in the report DB 8 may be referred to for reference. In this case, a search query corresponding to the medical image to be interpreted is used to search for past comments on findings described in the same or similar findings and/or diagnoses as the medical image to be interpreted. For example, in a case in which a nodule of a kidney is included in a medical image to be interpreted, a search is performed using “kidney” and “nodule” as a search query, and comments on findings including “kidney” and “nodule” are presented as search results.

In a case of searching for a comment on findings, it is desirable to use a search query as concise as possible so that a desired comment on findings can be quickly found. The reason for using a concise search query is, for example, to reduce the effort of a user to input the search query. In addition, for example, in a case in which the computer generates a search query by image analysis, this can shorten a processing time required to generate the search query and eliminate the need to develop an advanced image analysis function.

On the other hand, the more concise the search query, the greater the number and variations of comments on findings presented as search results, making it more difficult for a user to quickly find a desired comment on findings. Therefore, the information processing apparatus 10 according to the present embodiment provides a function of being able to search for an appropriate comment on findings with a concise search query so that a desired comment on findings can be quickly found even with a concise search query. The information processing apparatus 10 will be described below. As described above, the information processing apparatus 10 is encompassed in the interpretation WS 3.

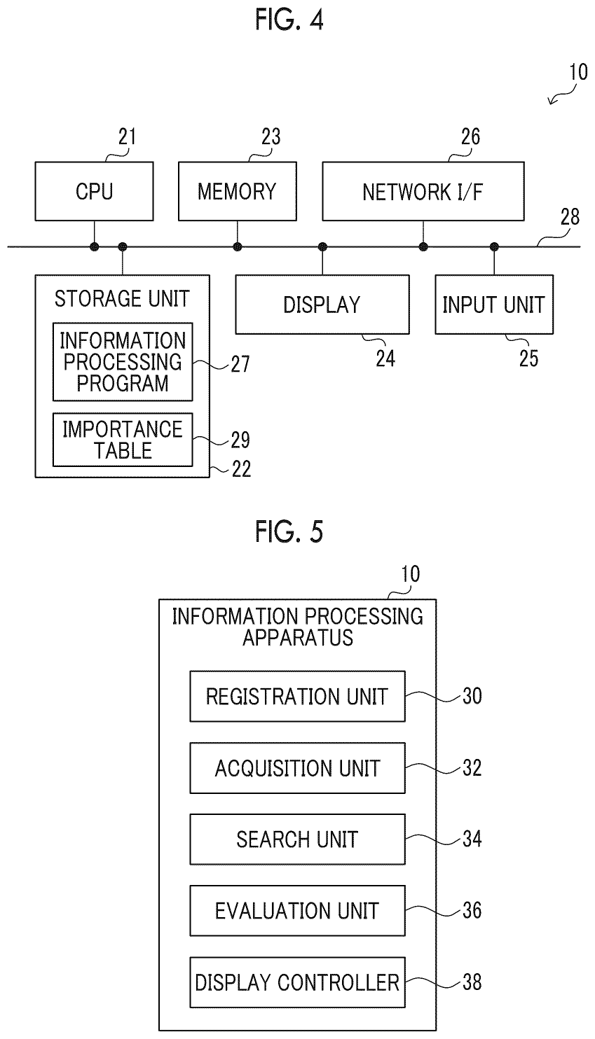

First, with reference to FIG. 4, an example of a hardware configuration of the information processing apparatus 10 according to the present embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired and/or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. Also, the storage unit 22 stores an importance table 29 (details will be described later). As the information processing apparatus 10, for example, a personal computer, a server computer, a smartphone, a tablet terminal, a wearable terminal, or the like can be applied as appropriate.

Next, with reference to FIGS. 5 to 9, an example of a functional configuration of the information processing apparatus 10 according to the present embodiment will be described. As shown in FIG. 5, the information processing apparatus 10 includes a registration unit 30, an acquisition unit 32, a search unit 34, an evaluation unit 36, and a display controller 38. In a case in which the CPU 21 executes the information processing program 27, the CPU 21 functions as each of the functional units, namely, a registration unit 30, an acquisition unit 32, a search unit 34, an evaluation unit 36, and a display controller 38.

### Structuring of Interpretation Report

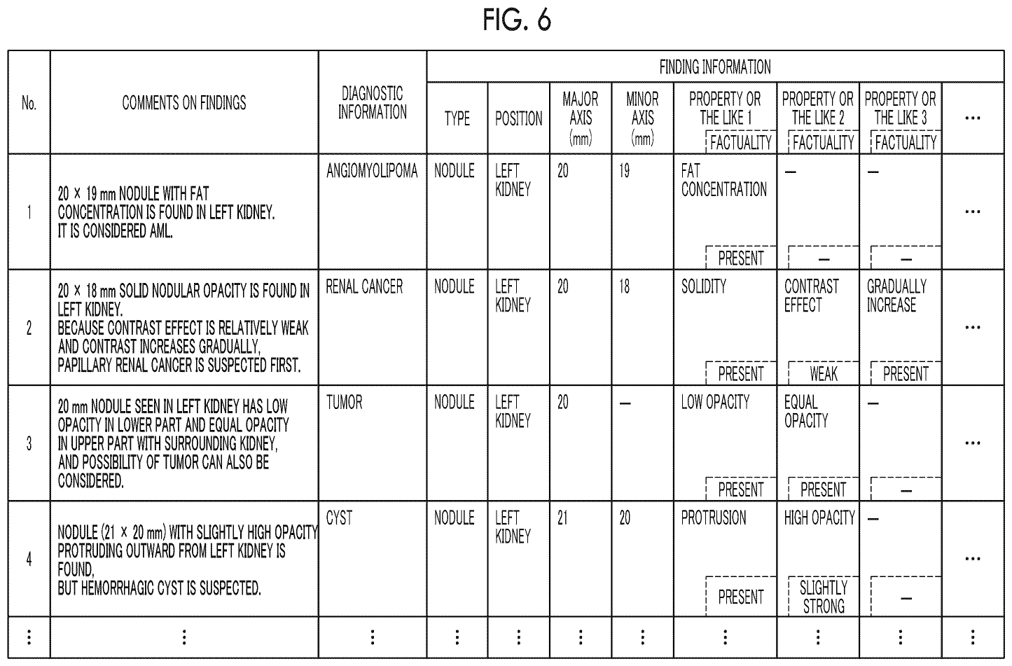

First, a process of structuring an interpretation report will be described as pre-processing executed by the information processing apparatus 10 prior to the process of searching for a comment on findings. As described above, in the report DB 8, created interpretation reports are registered. FIG. 6 shows an example of information included in an interpretation report registered in the report DB 8. As shown in FIG. 6, a plurality of comments on findings and diagnostic information and finding information included in each of the comments on findings are registered in the report DB 8 in association with each other.

The comments on findings are registered in the report DB 8 by the report server 7 at the time of creating each interpretation report. The registration unit 30 specifies, each of a plurality of comments on findings (hereinafter referred to as a comment-on-findings group) registered in the report DB 8, diagnostic information and finding information included in the comments on findings and adds the specified diagnostic information and finding information to the comments on findings (so-called structuring).

The diagnostic information is, for example, an estimated disease name diagnosed based on a medical image. The estimated disease name is an evaluation result estimated based on the lesion included in the medical image, and, for example, the disease name such as “cancer” and “inflammation” and the evaluation result such as “negative/positive”, “benign/malignant”, and “mild/severe” regarding disease names and properties can be mentioned. In FIG. 6, “angiomyolipoma”, “renal cancer”, “tumor”, and “cyst” are illustrated as examples of diagnostic information.

In addition, one comment on findings may include a plurality of estimated disease names, such as “renal cell carcinoma is suspected, but angiomyolipoma with a small fatty component may also be possible”. In this case, the registration unit 30 may select one estimated disease name as diagnostic information, and add other estimated disease names that were not selected to the comment on findings as finding information.

The finding information is, for example, information indicating at least one of various findings such as a type (name), a property, a position, a measurement value, and a number of a region of interest, a phrase expressing a change in the region of interest, and a type of diagnostic information other than the diagnostic information to be paired. As described above, the type of diagnostic information other than the diagnostic information to be paired is an estimated disease name or the like that was not selected as diagnostic information.

Examples of the type (name) of the region of interest include the names of structures, such as “lung” and “liver”, and the names of lesions, such as “nodule” and “ground glass opacity”. The property of the region of interest mainly means the features of the lesion. For example, in the case of a nodule, findings indicating opacity such as “solidity”, “low opacity”, and “high opacity”, margin shapes such as “well-defined/ill-defined”, “smooth/irregular”, “spicula”, “lobulated”, and “lagged”, and an overall shape such as “round” and “irregular form” can be mentioned. Also, for example, the relationship with the peripheral tissue, such as “protrusion”, and findings regarding the presence or absence of contrast, washout, and the like can be mentioned.

The position of a region of interest means an anatomical position, a position in a medical image, and a relative positional relationship with other regions of interest such as “inside”, “margin”, and “periphery”. The anatomical position may be expressed by organ names such as “lung” and “kidney”, or may be expressed by subdividing the kidney into “left kidney” and “superior segment”.

The measurement value of a region of interest is a value that can be quantitatively measured from a medical image, and is, for example, at least one of a size or a signal value of a region of interest. The size is represented by, for example, a major axis, a minor axis, an area, a volume, or the like of a region of interest. The signal value is represented by, for example, a pixel value in a region of interest, a CT value in units of HU, or the like. In addition, the finding information indicating the measurement value may be divided into predetermined classes (that is, quantized), such as “0 mm or more and less than 5 mm”, “5 mm or more and less than 10 mm”, and “10 mm or more and less than 15 mm”. In this case, for example, in a case in which the comment on findings includes the description “12 mm”, the finding information “10 mm or more and less than 15 mm” is specified. Furthermore, the finding information indicating the measurement value may simply be information indicating whether or not it is described in the comment on findings. This is because measurement values may have a large variation or may not be described.

The number of regions of interest may be expressed as a specific numerical value such as one or two, or may be expressed in relative expressions such as “single/multiple” and “few/many”. A phrase expressing a change in the region of interest is a phrase expressing changes over time in properties, positions, measurement values, numbers, and the like in a case in which follow-up observation for the region of interest is performed, and examples of such phrases include “appearance/disappearance”, “increase/reduction”, “worsening/improvement”, and “metastasis”.

Specifically, the registration unit 30 extracts named entities (words) from the comments on findings registered in the report DB 8, and specifies the diagnostic information or finding information corresponding to the extracted named entities. As a method for extracting named entities from a comment on findings, a known named entity extraction method using a natural language processing model such as, for example, bidirectional encoder representations from transformers (BERT) can be applied as appropriate.

In addition, the comment-on-findings group may include different words with the same meaning (synonyms), such as “angiomyolipoma” and “AML”. In order to deal with such spelling variations due to synonyms, in a case in which a comment-on-findings group includes synonyms, it is preferable that the registration unit 30 adds the same diagnostic information or finding information to each of the comments on findings including synonyms (so-called normalization).

Specifically, a dictionary in which a correspondence relationship between named entities that may be included in a comment on findings and diagnostic information or finding information is defined, in which synonymous named entities are associated with the same diagnostic information or finding information, may be stored in advance in the storage unit 22. For example, in the dictionary, “angiomyolipoma” and “AML” indicating synonyms may each be associated with the same diagnostic information, “angiomyolipoma”. The registration unit 30 may extract named entities from the comments on findings registered in the report DB 8 and specify the diagnostic information or finding information included in the comments on findings by referring to a dictionary.

In addition, it is preferable that the registration unit 30 also specifies the factuality of the specified diagnostic information or finding information. The “factuality” is information indicating whether the finding is found or not, and the degree of certainty thereof and the like. This is because interpretation reports may include not only findings that are clearly found from medical images, but also findings that are not found from medical images, and findings that are suspicious but have a low degree of certainty. For example, since the presence or absence and the degree of fatty components are used to differentiate between AML and renal cell carcinoma (RCC), the interpretation report may deliberately include that “no fatty components are observed”.

In addition, the finding information may be information that modifies other diagnostic information or finding information. In this case, it is preferable that the registration unit 30 also specifies a modification relationship between the finding information and the diagnostic information or between the pieces of finding information. For example, “calcification”, which is an example of the property of the lung nodule, may be described in detail as “microcalcification is observed in the center”. In this case, the registration unit 30 may specify the finding information of “center” and “micro” as other finding information that modifies the finding information of “calcification”. Examples of finding information that modifies “calcification” include “micro”, “coarse”, “scattered”, “center”, “ring-shaped”, and “complete”.

For example, in the case of “renal cell carcinoma”, which is an example of diagnostic information based on the nodule of the kidney, a tissue type such as “clear cell”, “papillary”, “chromophobe”, and “multilocular cystic” may also be described in the comments on findings. The registration unit 30 may specify the above-mentioned finding information indicating the tissue type as other finding information that modifies the finding information “renal cell carcinoma”.

Furthermore, in a case in which one comment on findings includes diagnostic information or finding information related to a plurality of regions of interest (lesions), it is preferable that the registration unit 30 specifies the diagnostic information or finding information for each region of interest.

Note that the comments on findings may not include diagnostic information. In this case, the registration unit 30 may add the diagnostic information “undiagnosed” to the comment on findings. For example, in a case in which a lesion (region of interest) included in a medical image is small and the findings can be interpreted but are below diagnostic criteria predetermined in the guidelines (that is, are not important), only the finding information may be described and not described as diagnostic information. Also, for example, in a case in which the region of interest is clearly benign, only the finding information may be described and not described as diagnostic information. In addition, for example, there are cases in which the creator of the comment on findings forgets to write it down, and cases in which the comment on findings cannot be specified due to the processing accuracy of the registration unit 30.

### Derivation of Importance

Next, a process of deriving importance using structured data registered in the report DB 8 will be described as pre-processing executed by the information processing apparatus 10 prior to the process of searching for comments on findings. By the above-described process of structuring the interpretation report, a plurality of comments on findings and diagnostic information and finding information included in each of the comments on findings are registered in the report DB 8 in association with each other.

The comment-on-findings group may include comments on findings with the same diagnosis but different findings, or comments on findings with the same findings but different diagnoses. For example, a typical example is that, in a case in which a nodule is seen in a medical image of the kidney, the nodule is diagnosed as AML in a case in which there is a fatty component, and the nodule is diagnosed as RCC in a case in which there is no fatty component, but in some cases, the nodule with a small fatty component may be diagnosed as AML.

For example, among the comment-on-findings group, some contain findings that are important for diagnosis, some contain comprehensive findings that include unimportant findings, and some even omit important findings. For example, there are comments on findings that simply state “A nodule suspected of AML is found”, while there are comments on findings that include finding information that serves as the basis for a diagnosis, such as comments on findings “A nodule suspected of AML is found, mainly consisting of fat concentration”.

It is considered appropriate for the comments on findings to be presented as search results to include all important findings without excess or deficiency. Therefore, the registration unit 30 derives the importance for each combination of diagnostic information and finding information as an index used for evaluating the comment on findings.

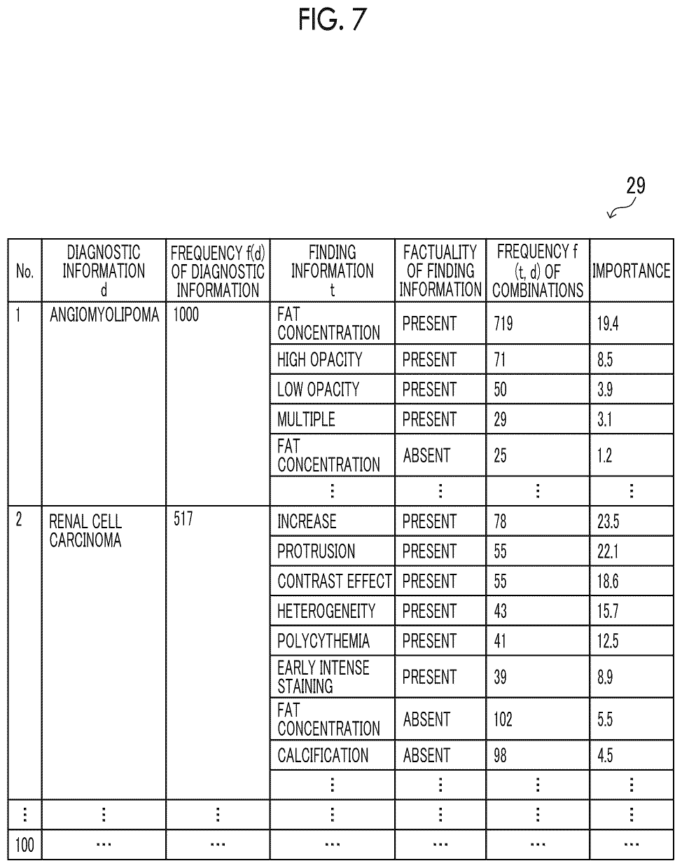

FIG. 7 shows an example of the importance table 29 in which the importance is determined for each combination of diagnostic information and finding information. The importance table 29 also indicates the frequency of diagnostic information included in the comment-on-findings group, and the frequency of combinations of diagnostic information and finding information included in the comment-on-findings group. In addition, the combinations are divided according to the factuality of the finding information.

From the example of FIG. 7, it can be seen that the comment-on-findings group includes 1000 comments on findings containing “angiomyolipoma” and 517 comments on findings containing “renal cell carcinoma”. It can also be seen that the comment-on-findings group includes 719 comments on findings containing the combination of “angiomyolipoma” and “fat concentration” being “present”. It can also be seen that the comment-on-findings group includes 25 comments on findings containing the combination of “angiomyolipoma” and “fat concentration” being “absent”. It can also be seen that the comment-on-findings group includes 102 comments on findings containing the combination of “renal cell carcinoma” and “fat concentration” being “absent”.

Specifically, the registration unit 30 may derive the importance based on a value corresponding to the frequency with which a combination of diagnostic information and finding information is included in a comment-on-findings groups. The more frequently a combination of diagnostic information and finding information is included in a comment-on-findings group, the more typical the combination is, and it is considered that the more likely it is that the combination will be desired by a user who performs a search.

For example, the registration unit 30 may derive the importance for each combination of diagnostic information d and finding information t based on the following Equation (1). f (d) is the frequency of diagnostic information d included in the comment-on-findings group, and f (t, d) is the frequency of the combination of diagnostic information d and finding information t included in the comment-on-findings group. According to Equation (1), it can be said that the more frequently a piece of finding information t is described in a comment on findings in combination with certain diagnostic information d, the higher its importance.

$$tf ( t , d ) = \frac{f ( t , d )}{f ( d )} \qquad ( 1 )$$

Furthermore, the registration unit 30 may derive the importance based on a value corresponding to the degree of uniqueness with which finding information included in a combination of certain diagnostic information and finding information is not included in combinations with other diagnostic information. The degree of uniqueness indicates whether certain finding information is so rare that it is used in combination with only some diagnostic information, or whether it is so common that it is used in combination with many diagnostic information. The more unique the finding information, the more valuable it is as a basis for a diagnosis, and it is considered that the more likely it is that the finding information will be desired by the user who performs the search.

For example, the registration unit 30 may derive the importance for each combination of diagnostic information d and finding information t based on the following Equation (2). N is the number of types of all pieces of diagnostic information D included in the comment-on-findings group, and nₜ is the number of types of diagnostic information that are included in the comment-on-findings group in combination with finding information t among all pieces of diagnostic information D. According to Equation (2), the fewer the number of types of diagnostic information described in combination with certain finding information t (that is, the higher the degree of uniqueness), the higher the importance is derived.

$$idf ( t , D ) = log ( \frac{N}{1 + {n}_{t}} ) + 1 \qquad ( 2 )$$

In addition, the registration unit 30 may derive the importance based on a value corresponding to the frequency with which a combination of diagnostic information and finding information is included in a comment-on-findings group and the degree of uniqueness with which the finding information included in the combination is not included in combinations with other diagnostic information. For example, the registration unit 30 may derive the importance based on the following Equation (3) which combines the two indices expressed by Equations (1) and (2). According to Equation (3), in a case in which both the frequency and the degree of uniqueness are high, high importance is derived.

$$tfidf ( t , d , D ) = tf ( t , d ) \cdot idf ( t , D ) \qquad ( 3 )$$

In the importance table 29 of FIG. 7, the corresponding value of Equation (1) for the combination of “angiomyolipoma” and “fat concentration” being “present” is derived as 0.719, since f (t, d) is 719 and f (d) is 1000.

In addition, in the importance table 29 of FIG. 7, the number of types N of all diagnostic information D is 100, and there is only one type of diagnostic information, “angiomyolipoma”, that can be combined with “fat concentration” being “present” (that is, nₜ is 1). In this case, the corresponding value of Equation (2) for the combination of “angiomyolipoma” and “fat concentration” being “present” is derived as log (100/(1+1))+1=2.70.

In the above case, the corresponding value of Equation (3) is derived as 0.719×2.70=1.94. In FIG. 7, as an example, a value obtained by multiplying the corresponding value in Equation (3) by 10 is shown as the importance.

The registration unit 30 may exclude, from the importance table 29, combinations of diagnostic information and finding information for which the derived importance is equal to or less than a predetermined threshold value, by regarding them as noise. Furthermore, the registration unit 30 may exclude, from the importance table 29, combinations of diagnostic information and finding information for which the derived importance is relatively low (for example, in the bottom 30%), by regarding them as noise.

### Search for Comments on Findings

Next, the process of searching for comments on findings executed by the information processing apparatus 10 will be described.

The acquisition unit 32 acquires a search query. For example, the acquisition unit 32 may receive a search query input by the user via the input unit 25. For example, the acquisition unit 32 may acquire a medical image to be interpreted from the image server 5, and generate at least one of finding information or diagnostic information about the medical image as a search query based on the medical image. In addition, a search query input by a user and a search query generated based on a medical image may be used in combination.

An example of a method for generating a search query based on a medical image is described. For example, the acquisition unit 32 extracts at least one region of interest (for example, a nodule region) included in the acquired medical image. As a method for extracting a region of interest, a method using known computer aided detection/diagnosis (CAD) technology and artificial intelligence (AI) technology can be applied as appropriate. For example, the acquisition unit 32 may extract a region of interest from a medical image by using a learning model such as a convolutional neural network (CNN) that has been trained to receive the medical image as an input and extract and output a region of interest included in the medical image.

The acquisition unit 32 then generates finding information and/or diagnostic information for the extracted region of interest. For example, the acquisition unit 32 may generate finding information and/or diagnostic information of a region of interest by using a learning model such as a CNN that has been trained in advance to receive the region of interest extracted from the medical image as an input and output the finding information and/or diagnostic information of the region of interest. The acquisition unit 32 may use at least one of the thus generated finding information and/or diagnostic information as a search query.

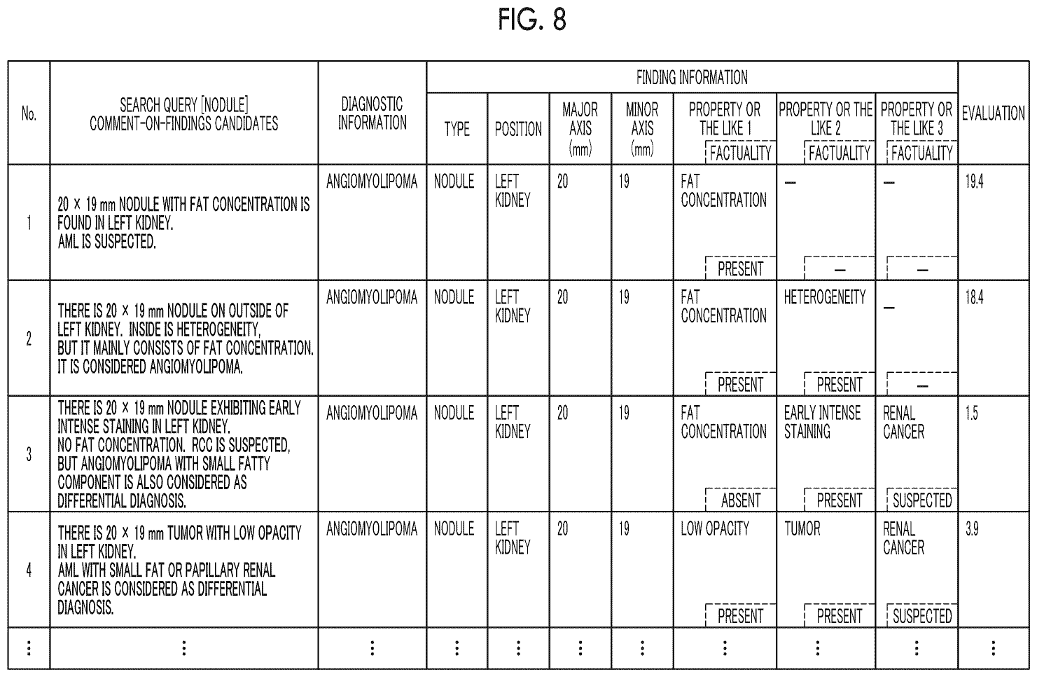

The search unit 34 searches for a plurality of comment-on-findings candidates related to the search query acquired by the acquisition unit 32 from a comment-on-findings group including a plurality of comments on findings registered in the report DB 8. FIG. 8 shows an example of a search result in a case in which “nodule” is used as a search query.

The evaluation unit 36 evaluates each of the comment-on-findings candidates searched for by the search unit 34 based on importance predetermined for each combination of the diagnostic information and the finding information included in the comment-on-findings candidates. As described above, the importance is predetermined by the registration unit 30 for each combination of diagnostic information and finding information included in the comment-on-findings group, and is stored in the storage unit 22 as the importance table 29. In the example of FIG. 8, the evaluation value derived by the evaluation unit 36 is shown in the “Evaluation” field.

Specifically, the evaluation unit 36 refers to the importance table 29 and acquires the importance corresponding to the combination of the diagnostic information and the finding information included in the comment-on-findings candidate. For example, the comment-on-findings candidate of No. 1 in FIG. 8 includes a combination of “angiomyolipoma” and “fat concentration” being “present”. In this case, the evaluation unit 36 acquires the importance “19.4” corresponding to the combination of “angiomyolipoma” and “fat concentration” being “present” from the importance table 29 in FIG. 7, and adds the acquired importance “19.4” as the evaluation value of the comment-on-findings candidate of No. 1 in FIG. 8.

In addition, in a case in which the comment-on-findings candidate includes a plurality of different combinations of diagnostic information and finding information, the evaluation unit 36 may perform the evaluation based on the importance of each combination. For example, the comment-on-findings candidate of No. 2 in FIG. 8 includes a first combination of “angiomyolipoma” and “fat concentration” being “present” and a second combination of “angiomyolipoma” and “heterogeneity” being “present”. In this case, the evaluation unit 36 may acquire the importance corresponding to each of the first combination and the second combination from the importance table 29 in FIG. 7, and may add representative values such as the total value, the average value, the weighted average value, and the multiplication value thereof as the evaluation values of the comment-on-findings candidates of No. 2 in FIG. 8.

Further, in a case in which the comment-on-findings candidate includes a combination of diagnostic information and finding information for which the importance is not predetermined, the evaluation unit 36 may correct the evaluation of the comment-on-findings candidate to be lowered. The combination of the diagnostic information and the finding information for which the importance is not predetermined is a combination that is not registered in the importance table 29 because it is not included in the comment-on-findings group or has a low frequency. Comment-on-findings candidates that include such combinations are considered to have low general-purpose properties, and therefore it is preferable to lower their evaluation. For example, in a case in which a comment-on-findings candidate includes a combination of diagnostic information and finding information that is not registered in the importance table 29, the evaluation unit 36 may subtract a predetermined value (for example, 1) from the evaluation value.

The display controller 38 presents at least one comment-on-findings candidate based on the result of the evaluation by the evaluation unit 36. For example, the display controller 38 may rearrange and present a plurality of comment-on-findings candidates in the order in which the evaluation values (that is, the importance) are evaluated as being high by the evaluation unit 36.

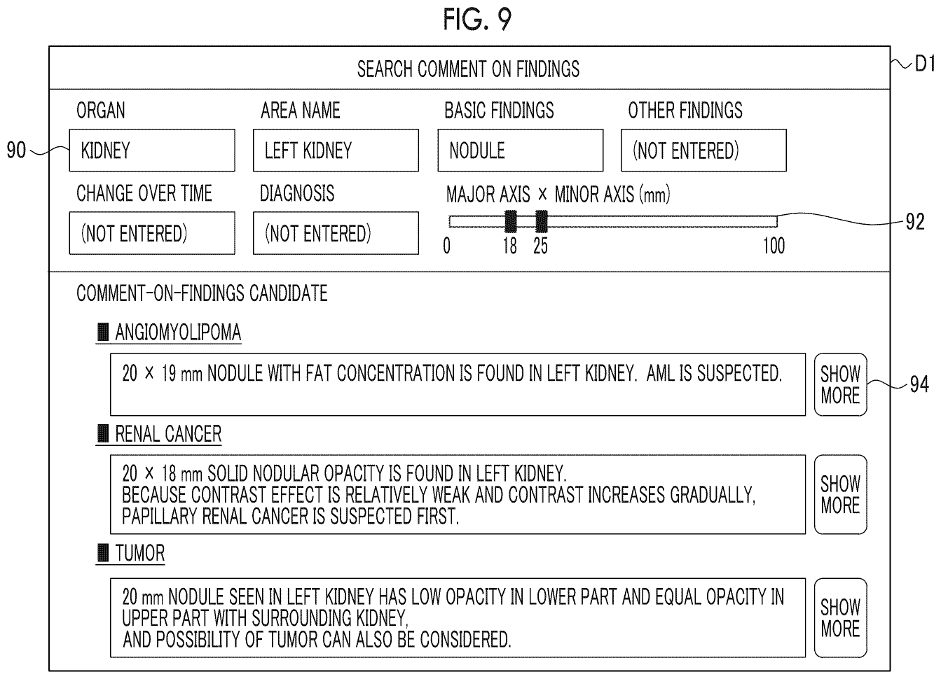

In addition, the display controller 38 may divide the plurality of comment-on-findings candidates into a plurality of groups based on the type of diagnostic information included in the comment-on-findings candidate and may present at least one comment-on-findings candidate for each group. FIG. 9 shows an example of a screen D1 for presenting the comment-on-findings candidate, which is displayed on the display 24 by the display controller 38. At the upper part of the screen D1, a text box 90 for the user to input a search query and a slider bar 92 for designating the major axis and minor axis of the region of interest are displayed. At the lower part of the screen D1, a plurality of comment-on-findings candidates searched for by the search unit 34 based on the input search query and evaluated by the evaluation unit 36 are displayed.

In the example of FIG. 9, one comment-on-findings candidate is presented for each of the three groups of “angiomyolipoma”, “renal cancer”, and “tumor”. In this way, by presenting a plurality of comment-on-findings candidates for each group, the user can check comment-on-findings candidates including a variety of diagnostic information.

Specifically, the display controller 38 may present one comment-on-findings candidate that is evaluated to have the highest importance by the evaluation unit 36 for each group. Further, for example, the display controller 38 may rearrange and present a plurality of comment-on-findings candidates in order of the importance evaluated as being high for each group.

In addition, in a case of presenting at least one comment-on-findings candidate for each group, the display controller 38 may rearrange the groups corresponding to the respective pieces of diagnostic information in descending order of the frequency with which the diagnostic information is included in the comment-on-findings group. That is, in the example of FIG. 9, the groups are arranged in the order of “angiomyolipoma”, “renal cancer”, and “tumor” from the top, but this order may be rearranged according to the frequency with which the diagnostic information is included in the comment-on-findings group. The frequency with which the diagnostic information is included in the comment-on-findings group is, for example, registered in advance in the importance table 29 (see FIG. 7). The more frequently diagnostic information is included in comment-on-findings group, the more commonly seen the case is, and it is considered that the more likely it is that the case will be desired by the user who performs the search.

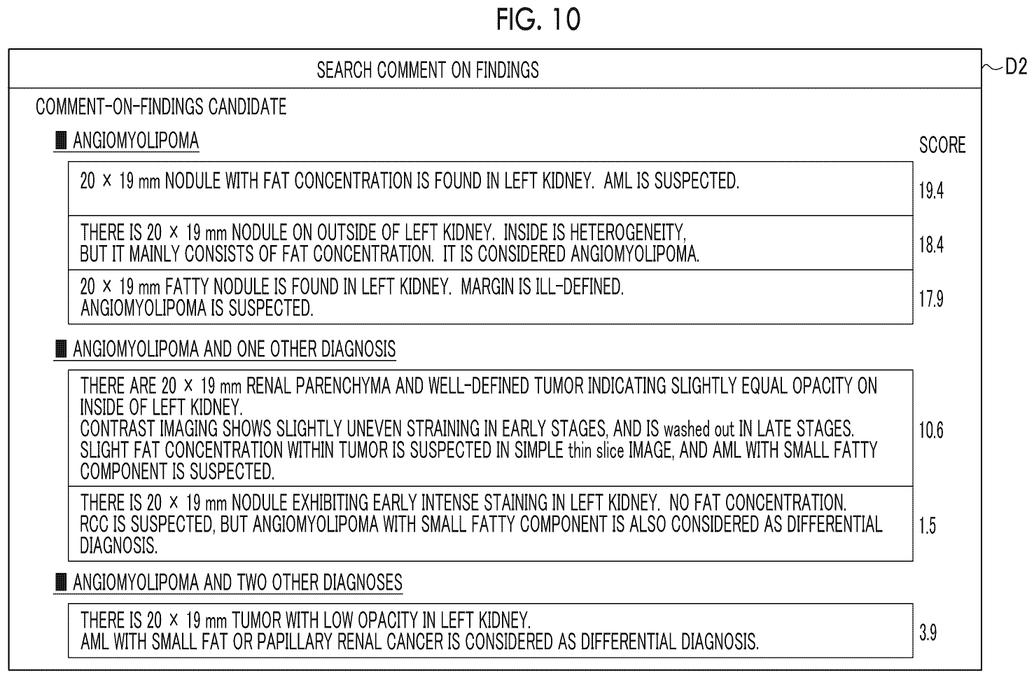

In addition, the display controller 38 may further divide the comment-on-findings candidates included in the groups into subgroups based on a predetermined criterion, and may present at least one comment-on-findings candidate for each of the subgroups. FIG. 10 shows an example of a screen D2 for presenting comment-on-findings candidates, which is transition to and displayed on the display 24 by the display controller 38 in a case in which the “show more” button 94 for “angiomyolipoma” is selected on the screen D1. On the screen D2, a plurality of comment-on-findings candidates for the selected “angiomyolipoma” are displayed. Furthermore, on the screen D2, the evaluation value (importance) derived by the evaluation unit 36 is displayed as a “score”.

In the example of FIG. 10, the comment-on-findings candidates are presented for each of the three groups of “angiomyolipoma”, “angiomyolipoma and one other diagnosis” and “angiomyolipoma and two other diagnoses”. In this way, the display controller 38 may divide the comment-on-findings candidates into subgroups using the number of types of diagnostic information included in the comment-on-findings candidates as a predetermined criterion. In general, the more diagnostic information included in a comment on findings, the more vague the comment on findings becomes, so that it is preferable to present subgroups with a smaller number of pieces of diagnostic information at a higher rank.

The criteria used for subgrouping are not limited to the number of types of diagnostic information described above. For example, the display controller 38 may divide a plurality of comment-on-findings candidates into subgroups based on the factuality of diagnostic information included in the comment-on-findings candidates. For example, comment-on-findings candidates may be presented for each subgroup according to the factuality, such as “highly likely”, “suspected”, “possible”, “cannot be denied”, “cannot be differentiated”, and “negative” for “angiomyolipoma”.

In addition, in a case in which there are a plurality of comment-on-findings candidates that include the same combination and have the same result of the evaluation, the display controller 38 may select and present any one of the comment-on-findings candidates. For example, in a case in which there are a plurality of comment-on-findings candidates that include the same combination and have the same evaluation result, the display controller 38 may select and present one having the smallest sentence volume among the comment-on-findings candidates. It is considered that the comment-on-findings candidates to be presented as search results preferably include a variety of contents. Therefore, the display controller 38 may be configured to prevent a plurality of comment-on-findings candidates having the same content from being presented.

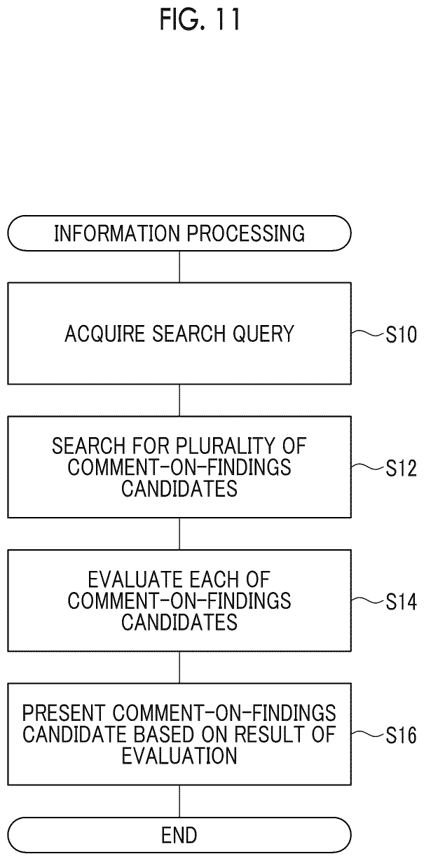

Next, with reference to FIG. 11, operations of the information processing apparatus 10 according to the embodiment will be described. In the information processing apparatus 10, as the CPU 21 executes the information processing program 27, information processing shown in FIG. 11 is executed. The information processing is executed, for example, in a case in which the user gives an instruction to start execution via the input unit 25. It is assumed that the process of structuring the interpretation report and the process of deriving the importance described above have been executed in advance.

In Step S10, the acquisition unit 32 acquires a search query. In Step S12, the search unit 34 searches for a plurality of comment-on-findings candidates related to the search query acquired in Step S10 from a comment-on-findings group including a plurality of comments on findings registered in the report DB 8.

In Step S14, the evaluation unit 36 evaluates each of the comment-on-findings candidates searched for in Step S12 based on importance predetermined (for example, the importance registered in the importance table 29) for each combination of diagnostic information and finding information included in the comment-on-findings candidates. In Step S16, the display controller 38 performs control to display a screen for presenting at least one comment-on-findings candidate on the display 24 based on the result of the evaluation in Step S14, and then ends the present information processing.

As described above, the information processing apparatus 10 according to one aspect of the present disclosure comprises at least one processor. The processor searches for a plurality of comment-on-findings candidates related to a search query from a comment-on-findings group including a plurality of comments on findings, evaluates each of the comment-on-findings candidates based on importance predetermined for each combination of diagnostic information and finding information included in the comment-on-findings candidates, and presents at least one of the comment-on-findings candidates based on a result of the evaluation.

That is, with the information processing apparatus 10 according to the present embodiment, since the comment-on-findings candidate having a high importance can be preferentially presented, it is possible to search for an appropriate comment on findings with a concise search query. Therefore, it is possible to reduce the effort of the user to input the search query and quickly present the comments on findings. In addition, even in a case in which a computer generates a search query using a CAD technology, it is only necessary to generate a concise search query, so that it is possible to shorten the processing time required to generate the search query and eliminate the need to develop an advanced image analysis function.

Further, in each of the above embodiments, a form assuming interpretation for medical images has been described, but the present disclosure is not limited thereto. The information processing apparatus 10 of the present disclosure can be applied to various images including a region of interest, which are obtained by imaging a subject. For example, the information processing apparatus 10 may be applied to an image acquired using an apparatus, a building, a pipe, a welded portion, or the like as a subject in a non-destructive examination such as a radiation transmission examination and an ultrasonic flaw detection examination. In this case, for example, the region of interest indicates cracks, flaws, bubbles, foreign matter, or the like.

In addition, in each of the above embodiments, for example, as hardware structures of processing units that execute various kinds of processing, such as the registration unit 30, the acquisition unit 32, the search unit 34, the evaluation unit 36, and the display controller 38, various processors shown below can be used. As described above, the various processors include a programmable logic device (PLD) as a processor of which the circuit configuration can be changed after manufacture, such as a field-programmable gate array (FPGA), a dedicated electrical circuit as a processor having a dedicated circuit configuration for executing specific processing such as an application-specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system-on-chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 27 is described as being stored (installed) in the storage unit 22 in advance; however, the present disclosure is not limited thereto. The information processing program 27 may be provided in a form recorded in a recording medium such as a compact disc read-only memory (CD-ROM), a digital versatile disc read-only memory (DVD-ROM), and a universal serial bus (USB) memory. In addition, the information processing program 27 may be configured to be downloaded from an external device via a network. Further, the technology of the present disclosure extends to a storage medium for storing the information processing program non-transitorily in addition to the information processing program.

The technology of the present disclosure can be appropriately combined with the above-described embodiment and examples. The described contents and illustrated contents shown above are detailed descriptions of the parts related to the technology of the present disclosure, and are merely an example of the technology of the present disclosure. For example, the above description of the configuration, function, operation, and effect is an example of the configuration, function, operation, and effect of the parts related to the technology of the present disclosure. Therefore, needless to say, unnecessary parts may be deleted, new elements may be added, or replacements may be made to the described contents and illustrated contents shown above within a range that does not deviate from the gist of the technology of the present disclosure.

Regarding the embodiments, the following supplementary notes are further disclosed.

### Supplementary Note 1

An information processing apparatus comprising at least one processor,
in which the processor is configured to:
   search for a plurality of comment-on-findings candidates related to a search query from a comment-on-findings group including a plurality of comments on findings;
   evaluate each of the comment-on-findings candidates based on importance predetermined for each combination of diagnostic information and finding information included in the comment-on-findings candidates; and
   present at least one of the comment-on-findings candidates based on a result of the evaluation.

### Supplementary Note 2

The information processing apparatus according to Supplementary Note 1,
in which the processor is configured to rearrange and present the plurality of comment-on-findings candidates in order of the importance evaluated as being high.

### Supplementary Note 3

The information processing apparatus according to Supplementary Note 1 or 2,
in which the processor is configured to:
   divide the plurality of comment-on-findings candidates into a plurality of groups based on a type of the diagnostic information included in the comment-on-findings candidates; and
   present, for each group, at least one of the comment-on-findings candidates.

### Supplementary Note 4

The information processing apparatus according to Supplementary Note 3,
in which the processor is configured to rearrange and present, for each group, the plurality of comment-on-findings candidates in order of the importance evaluated as being high.

### Supplementary Note 5

The information processing apparatus according to Supplementary Note 3,
in which the processor is configured to present, for each group, one of the comment-on-findings candidates that is evaluated as having the highest importance.

### Supplementary Note 6

The information processing apparatus according to any one of Supplementary Notes 3 to 5,
in which the processor is configured to:
   further divide the comment-on-findings candidates included in the groups into subgroups based on a predetermined criterion; and
   present, for each subgroup, at least one of the comment-on-findings candidates.

### Supplementary Note 7

The information processing apparatus according to Supplementary Note 6,
in which the predetermined criterion is at least one of the number of types of the diagnostic information included in the comment-on-findings candidates or factuality of the diagnostic information included in the comment-on-findings candidates.

### Supplementary Note 8

The information processing apparatus according to any one of Supplementary Notes 3 to 7,
in which the processor is configured to rearrange the groups corresponding to the respective pieces of diagnostic information in descending order of a frequency with which the diagnostic information is included in the comment-on-findings group.

### Supplementary Note 9

The information processing apparatus according to any one of Supplementary Notes 1 to 8,
in which the importance is a value corresponding to a frequency with which the combination is included in the comment-on-findings group.

### Supplementary Note 10

The information processing apparatus according to any one of Supplementary Notes 1 to 8,
in which the importance is a value corresponding to a degree of uniqueness with which the finding information included in the combination is not included in combinations with other diagnostic information.

### Supplementary Note 11

The information processing apparatus according to any one of Supplementary Notes 1 to 8,
in which the importance is a value corresponding to a frequency with which the combination is included in the comment-on-findings group and a degree of uniqueness with which the finding information included in the combination is not included in combinations with other diagnostic information.

### Supplementary Note 12

The information processing apparatus according to any one of Supplementary Notes 1 to 11,
in which the processor is configured to, in a case in which the comment-on-findings candidates include a plurality of different combinations, perform the evaluation based on the importance for each of the combinations.

### Supplementary Note 13

The information processing apparatus according to any one of Supplementary Notes 1 to 12,
in which the importance is predetermined for each combination included in the comment-on-findings group, and
the processor is configured to, in a case in which the comment-on-findings candidate includes the combination for which the importance is not predetermined, correct the evaluation of the comment-on-findings candidate to be lowered.

### Supplementary Note 14

The information processing apparatus according to any one of Supplementary Notes 1 to 13,
in which the processor is configured to, in a case in which there are a plurality of the comment-on-findings candidates that include the same combination and have the same result of the evaluation, select and present any one of the comment-on-findings candidates.

### Supplementary Note 15

The information processing apparatus according to Supplementary Note 14,
in which the processor is configured to, in a case in which there are a plurality of the comment-on-findings candidates that include the same combination and have the same result of the evaluation, select and present one having the smallest sentence volume among the comment-on-findings candidates.

### Supplementary Note 16

The information processing apparatus according to any one of Supplementary Notes 1 to 15,
in which the processor is configured to receive an input of the search query by a user.

### Supplementary Note 17

The information processing apparatus according to any one of Supplementary Notes 1 to 16,
in which the processor is configured to:
   acquire an image; and
   generate at least one of the finding information or the diagnostic information for the image as the search query based on the image.

### Supplementary Note 18

The information processing apparatus according to any one of Supplementary Notes 1 to 17,
in which the finding information indicates at least one of a type, a property, a position, a measurement value, and a number of a region of interest, a phrase expressing a change in the region of interest, or a type of diagnostic information other than the diagnostic information to be paired.

### Supplementary Note 19

An information processing method executed by a computer, the information processing method comprising:
searching for a plurality of comment-on-findings candidates related to a search query from a comment-on-findings group including a plurality of comments on findings;
evaluating each of the comment-on-findings candidates based on importance predetermined for each combination of diagnostic information and finding information included in the comment-on-findings candidates; and
presenting at least one of the comment-on-findings candidates based on a result of the evaluation.

### Supplementary Note 20

An information processing program for causing a computer to execute:
searching for a plurality of comment-on-findings candidates related to a search query from a comment-on-findings group including a plurality of comments on findings;
evaluating each of the comment-on-findings candidates based on importance predetermined for each combination of diagnostic information and finding information included in the comment-on-findings candidates; and
presenting at least one of the comment-on-findings candidates based on a result of the evaluation.

What is claimed is:

1. An information processing apparatus comprising at least one processor, wherein the processor is configured to:
search for a plurality of comment-on-findings candidates related to a search query from a comment-on-findings group including a plurality of comments on findings;
evaluate each of the comment-on-findings candidates based on importance predetermined for each combination of diagnostic information and finding information included in the comment-on-findings candidates; and
present at least one of the comment-on-findings candidates based on a result of the evaluation.

2. The information processing apparatus according to claim 1, wherein the processor is configured to rearrange and present the plurality of comment-on-findings candidates in order of the importance evaluated as being high.

3. The information processing apparatus according to claim 1, wherein the processor is configured to:
divide the plurality of comment-on-findings candidates into a plurality of groups based on a type of the diagnostic information included in the comment-on-findings candidates; and
present, for each group, at least one of the comment-on-findings candidates.

4. The information processing apparatus according to claim 3, wherein the processor is configured to rearrange and present, for each group, the plurality of comment-on-findings candidates in order of the importance evaluated as being high.

5. The information processing apparatus according to claim 3, wherein the processor is configured to present, for each group, one of the comment-on-findings candidates that is evaluated as having a highest importance.

6. The information processing apparatus according to claim 3, wherein the processor is configured to:
further divide the comment-on-findings candidates included in the groups into subgroups based on a predetermined criterion; and
present, for each subgroup, at least one of the comment-on-findings candidates.

7. The information processing apparatus according to claim 6, wherein the predetermined criterion is at least one of the number of types of the diagnostic information included in the comment-on-findings candidates or factuality of the diagnostic information included in the comment-on-findings candidates.

8. The information processing apparatus according to claim 3, wherein the processor is configured to rearrange the groups corresponding to the respective pieces of diagnostic information in descending order of a frequency with which the diagnostic information is included in the comment-on-findings group.

9. The information processing apparatus according to claim 1, wherein the importance is a value corresponding to a frequency with which the combination is included in the comment-on-findings group.

10. The information processing apparatus according to claim 1, wherein the importance is a value corresponding to a degree of uniqueness with which the finding information included in the combination is not included in combinations with other diagnostic information.

11. The information processing apparatus according to claim 1, wherein the importance is a value corresponding to:
a frequency with which the combination is included in the comment-on-findings group; and
a degree of uniqueness with which the finding information included in the combination is not included in combinations with other diagnostic information.

12. The information processing apparatus according to claim 1, wherein the processor is configured to, in a case in which the comment-on-findings candidates include a plurality of different combinations, perform the evaluation based on the importance for each of the combinations.

13. The information processing apparatus according to claim 1, wherein:
the importance is predetermined for each combination included in the comment-on-findings group, and
the processor is configured to, in a case in which the comment-on-findings candidate includes the combination for which the importance is not predetermined, correct the evaluation of the comment-on-findings candidate to be lowered.

14. The information processing apparatus according to claim 1, wherein the processor is configured to, in a case in which there are a plurality of the comment-on-findings candidates that include the same combination and have the same result of the evaluation, select and present any one of the comment-on-findings candidates.

15. The information processing apparatus according to claim 14, wherein the processor is configured to, in a case in which there are a plurality of the comment-on-findings candidates that include the same combination and have the same result of the evaluation, select and present one having a smallest sentence volume among the comment-on-findings candidates.

16. The information processing apparatus according to claim 1, wherein the processor is configured to receive an input of the search query by a user.

17. The information processing apparatus according to claim 1, wherein the processor is configured to:
acquire an image; and
generate at least one of the finding information or the diagnostic information for the image as the search query based on the image.

18. The information processing apparatus according to claim 1, wherein the finding information indicates at least one of a type, a property, a position, a measurement value, and a number of a region of interest, a phrase expressing a change in the region of interest, or a type of diagnostic information other than the diagnostic information to be paired.

19. An information processing method executed by a computer, the information processing method comprising:
searching for a plurality of comment-on-findings candidates related to a search query from a comment-on-findings group including a plurality of comments on findings;
evaluating each of the comment-on-findings candidates based on importance predetermined for each combination of diagnostic information and finding information included in the comment-on-findings candidates; and
presenting at least one of the comment-on-findings candidates based on a result of the evaluation.

20. An information processing program for causing a computer to execute:
searching for a plurality of comment-on-findings candidates related to a search query from a comment-on-findings group including a plurality of comments on findings;
evaluating each of the comment-on-findings candidates based on importance predetermined for each combination of diagnostic information and finding information included in the comment-on-findings candidates; and
presenting at least one of the comment-on-findings candidates based on a result of the evaluation.
